(19) United States
(12) Patent Application Publication
(10) Pub. No.: US 2024/0078664 A1
(43) Pub. Date: Mar. 7, 2024

(54) ULTRASONIC IMAGING APPARATUS AND PROGRAM

(71) Applicant: FUJIFILM Healthcare Corporation, Kashiwa-shi (JP)

(72) Inventors: Teiichiro Ikeda, Chiba (JP); Takehiro Tsujita, Chiba (JP); Atsuko Otake, Chiba (JP); Atsushi Shiromaru, Chiba (JP); Rei Yokosawa, Chiba (JP)

(21) Appl. No.: 18/238,689

(22) Filed: Aug. 28, 2023

(30) Foreign Application Priority Data
Sep. 6, 2022 (JP) 2022-141600

(51) Int. Cl. G06T 7/00; G16H 30/20; G16H 30/40

(52) U.S. Cl. CPC G06T 7/0012; G16H 30/20; G16H 30/40; G06T 2207/10132; G06T 2207/30056; G06T 2207/30084

(57) ABSTRACT

An accuracy calculation unit receives an ultrasound image generated by transmitting and receiving an ultrasonic wave, and specifies an imaging scene by a plurality of types of specifying processing for specifying the imaging scene of the ultrasound image. Further, the accuracy calculation unit calculates accuracy of specifying the imaging scene for each specifying processing. The determination unit determines an examination action to be performed next on the basis of a result of calculation by the accuracy calculation unit.

### CROSS REFERENCE TO RELATED APPLICATION

This application claims priority to Japanese Patent Application No. 2022-141600 filed on Sep. 6, 2022, which is incorporated herein by reference in its entirety including the specification, claims, drawings, and abstract.

### TECHNICAL FIELD

The present disclosure relates to an ultrasonic imaging apparatus, and particularly relates to a technique for supporting an examiner.

### BACKGROUND

In a case where an examination is performed using an ultrasonic imaging apparatus, in general, the examiner such as a technician or a doctor performs the examination while determining in real time an examination action to be performed next in an examination workflow.

JP 2020-068797 A describes an apparatus for extracting a cross-sectional image of a target cross-section using multi-scale learning data.

JP 2010-279499 A describes an apparatus for detecting an MPR image necessary for stress echo examination from three-dimensional image data and detecting another necessary MPR image based on the detected MPR image.

JP 2014-184341 A discloses an apparatus for identifying a position of interest M by marking on a three-dimensional ultrasound image.

Meanwhile, in order to improve workflow of ultrasonic examination and reduce a burden on the examiner, it is effective that the ultrasonic imaging apparatus supports the examiner. However, conventionally, only recognition of an examination cross-section or only extraction of a region is performed, and the entire workflow of the ultrasonic examination is not optimized.

An object of the present disclosure is to improve the workflow of the ultrasonic examination using the ultrasonic imaging apparatus.

### SUMMARY

One aspect of the present disclosure is an ultrasonic imaging apparatus including: an accuracy calculation unit configured to receive an ultrasound image generated by transmitting and receiving an ultrasonic wave, to specify an imaging scene by a plurality of types of specifying processing for specifying the imaging scene of the ultrasound image, and to calculate accuracy of specifying the imaging scene for each specifying processing; and a determination unit configured to determine an examination action to be performed next on the basis of a result of calculation by the accuracy calculation unit.

According to the above configuration, the imaging scene is specified by the plurality of types of specifying processing, and the examination action to be performed next is determined on the basis of a result of specifying the imaging scene and the accuracy. Thus, the entire workflow of the ultrasonic examination can be optimized as compared with a case where only the recognition of the examination cross-section or only the extraction of the region is performed. That is, by specifying the imaging scene by the plurality of types of specifying processing, the accuracy of specifying the imaging scene is increased as compared with a case where the imaging scene is specified by one type of specifying processing. As a result, it is possible to improve accuracy of determination of the examination action to be performed next.

The plurality of types of specifying processing may include identification processing of a cross-section in which the ultrasonic wave is transmitted and received. The accuracy calculation unit may compare an image of a predetermined standard cross-section with an image of the cross-section in which the ultrasonic wave is transmitted and received to identify the cross-section in which the ultrasonic wave is transmitted and received, and may calculate accuracy of identifying the cross-section as the accuracy of specifying the imaging scene.

The plurality of types of specifying processing may further include processing of detecting an abnormality shown in the ultrasound image. The accuracy calculation unit may further detect the abnormality from the ultrasound image and calculate accuracy of detecting the abnormality as the accuracy of specifying the imaging scene.

The plurality of types of specifying processing may further include processing of specifying a site shown in the ultrasound image. The accuracy calculation unit may further specify the site shown in the ultrasound image and calculate the accuracy of specifying the site as the accuracy of specifying the imaging scene.

At least one of the result of the calculation by the accuracy calculation unit and a result determined on the basis of the result of the calculation may be used to determine the examination action to be performed next.

The determination unit may determine setting of a body mark to be set in the ultrasound image as the examination action to be performed next.

The determination unit may determine attachment of the ultrasound image on a report as the examination action to be performed next.

One aspect of the present disclosure is a computer-readable recording medium recording a program for causing a computer to function as: an accuracy calculation unit configured to receive an ultrasound image generated by transmitting and receiving an ultrasonic wave, to specify an imaging scene by a plurality of types of specifying processing for specifying the imaging scene of the ultrasound image, and to calculate accuracy of specifying the imaging scene for each specifying processing; and a determination unit configured to determine an examination action to be performed next on the basis of a result of calculation by the accuracy calculation unit.

According to the present disclosure, it is possible to improve the workflow of the ultrasonic examination using the ultrasonic imaging apparatus.

### BRIEF DESCRIPTION OF THE DRAWINGS

FIG. 1 is a block diagram illustrating a configuration of an ultrasonic imaging apparatus according to an embodiment;

FIG. 2 is a block diagram illustrating a configuration of an analysis unit according to the embodiment;

FIG. 3 is a graph illustrating accuracy of recognizing an examination cross-section;

FIG. 4 is a diagram illustrating a display example of accuracy;

FIG. 5 is a diagram illustrating a display example of accuracy;

FIG. 6 is a diagram illustrating a display example of accuracy;

FIG. 7 is a diagram illustrating a display example of accuracy;

FIG. 8 is a diagram illustrating a display example of accuracy;

FIG. 9 is a diagram illustrating a display example of an ultrasound image;

FIG. 10 is a diagram illustrating a display example of the ultrasound image;

FIG. 11 is a diagram illustrating a display example of the ultrasound image;

FIG. 12 is a diagram illustrating a body mark;

FIG. 13 is a diagram illustrating a plurality of ultrasound images;

FIG. 14 is a diagram illustrating the plurality of ultrasound images;

FIG. 15 is a diagram illustrating a display example of the ultrasound image;

FIG. 16 is a diagram illustrating a display example of a report;

FIG. 17 is a diagram illustrating the plurality of ultrasound images; and

FIG. 18 is a diagram illustrating a display example of the report.

### DESCRIPTION OF EMBODIMENTS

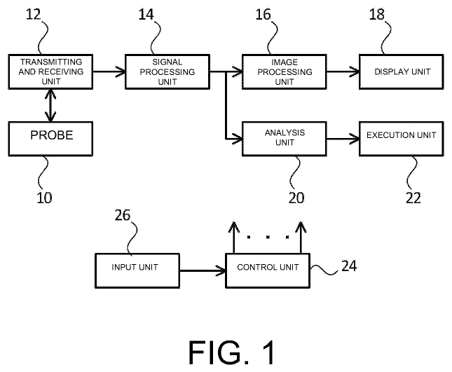

An ultrasonic imaging apparatus according to an embodiment will be described with reference to FIG. 1. FIG. 1 illustrates a configuration of the ultrasonic imaging apparatus according to the embodiment.

The ultrasonic imaging apparatus generates image data by transmitting and receiving an ultrasonic wave using an ultrasonic probe 10. For example, the ultrasonic imaging apparatus transmits the ultrasonic wave into a subject and receives the ultrasonic wave reflected inside the subject, thereby generating ultrasound image data representing tissue inside the subject.

The ultrasonic probe 10 is a device that transmits and receives the ultrasonic wave. The ultrasonic probe 10 includes, for example, a 1D array transducer. The 1D array transducer includes a plurality of ultrasonic transducers arranged one-dimensionally. An ultrasonic beam is formed by the 1D array transducer, and the ultrasonic beam is repeatedly electronically scanned. Thus, a scanning surface is formed in a living body for each electronic scanning. The scanning surface corresponds to a two-dimensional echo data acquisition space. In addition, an operation surface may be formed by electronic operation using a 1.25D array transducer, a 1.5D array transducer, or a 1.75D array transducer, which gives a degree of freedom in a minor axis direction of the 1D array transducer. The ultrasonic probe 10 may include a 2D array transducer formed by a plurality of vibration elements arranged two-dimensionally instead of the 1D array transducer. When the ultrasonic beam is formed by the 2D array transducer and is repeatedly electronically scanned, the scanning surface as the two-dimensional echo data acquisition space is formed for each electronic scanning. When the ultrasonic beam is two-dimensionally scanned, a three-dimensional space as a three-dimensional echo data acquisition space is formed. As a scanning method, sector scanning, linear scanning, convex scanning, or the like is used. In addition, an end fire-type probe used in an intravascular ultrasound (IVUS), an endoscopic ultrasound (EUS), or the like, or a probe including a single plate element may be used. The scanning surface may be formed by radial scanning.

A transmitting and receiving unit 12 functions as a transmission beamformer and a reception beamformer. At the time of transmission, the transmitting and receiving unit 12 supplies a plurality of transmitting signals having a certain delay relationship to the plurality of ultrasonic transducers included in the ultrasonic probe 10. Thus, an ultrasonic transmission beam is formed. At the time of reception, a reflected wave (an RF signal) from an inside of the living body is received by the ultrasonic probe 10, whereby a plurality of reception signals are output from the ultrasonic probe 10 to the transmitting and receiving unit 12. The transmitting and receiving unit 12 forms a reception beam by applying phasing addition processing to the plurality of reception signals. Beam data of the reception beam are output to a signal processing unit 14. That is, the transmitting and receiving unit 12 performs delay processing on the reception signal obtained from each ultrasonic transducer according to a delay processing condition for each ultrasonic transducer, and performs addition processing on the plurality of reception signals obtained from the plurality of ultrasonic transducers, thereby forming the reception beam. The delay processing condition is defined by reception delay data indicating a delay time. A reception delay data set (that is, a set of delay times) corresponding to the plurality of ultrasonic transducers is supplied from a control unit 24.

By an action of the transmitting and receiving unit 12, the ultrasonic beam (that is, the transmission beam and the reception beam) is electronically scanned, thereby forming the scanning surface. The scanning surface corresponds to a plurality of sets of beam data, and they constitute received frame data (specifically, RF signal frame data). Note that each set of beam data includes a plurality of sets of echo data arranged in a depth direction. By repeating the electronic scanning of the ultrasonic beam, a plurality of sets of received frame data arranged on a time axis are output from the transmitting and receiving unit 12 to the signal processing unit 14. The plurality of sets of received frame data constitute a received frame sequence.

When the ultrasonic beam is two-dimensionally electronically scanned by the action of the transmitting and receiving unit 12, the three-dimensional echo data acquisition space is formed, and volume data as an echo data aggregate are acquired from the three-dimensional echo data acquisition space. By repeating the electronic scanning of the ultrasonic beam, a plurality of sets of volume data arranged on the time axis are output from the transmitting and receiving unit 12 to the signal processing unit 14. The plurality of sets of volume data constitutes a volume data sequence.

The signal processing unit 14 generates the ultrasound image data (for example, B-mode image data) by applying signal processing such as amplitude compression (amplitude conversion) such as detection and logarithmic compression and a conversion function (a coordinate conversion function, an interpolation processing function, and the like performed by a digital scan converter (DSC)) to the beam data output from the transmitting and receiving unit 12. Hereinafter, the image data are appropriately referred to as an “image”. For example, the ultrasound image data are appropriately referred to as an “ultrasound image”, and the B-mode image data are appropriately referred to as a “B-mode image”. Note that the ultrasound image according to the present embodiment is not limited to the B-mode image, and may be any image data generated by an ultrasonic diagnosis apparatus. For example, the ultrasound image according to the present embodiment may be a color Doppler image, a pulse Doppler image, a strain image, a shear wave elastography image, or the like.

An image processing unit 16 overlays necessary graphic data on the ultrasound image data to generate display image data. The display image data are output to a display unit 18, and one or more images are displayed side by side in a display mode according to the display mode.

The display unit 18 is a display such as a liquid crystal display or an EL display. The ultrasound image such as the B-mode image is displayed on the display unit 18. The display unit 18 may be a device having both a display and an input unit 26. For example, a graphic user interface (GUI) may be implemented by the display unit 18. Further, a user interface such as a touch panel may be implemented by the display unit 18.

An analysis unit 20 receives the ultrasound image generated by transmitting and receiving the ultrasonic wave, and applies a plurality of types of specifying processing for specifying the imaging scene of the ultrasound image to the ultrasound image to specify the imaging scene. For example, the analysis unit 20 specifies the imaging scene for each specifying processing. Further, the analysis unit 20 calculates accuracy of specifying the imaging scene for each specifying processing. Furthermore, the analysis unit 20 determines an examination action to be performed next on the basis of a result of the specifying processing.

The workflow of the ultrasonic examination (that is, examination protocol or examination procedure) includes a plurality of examination actions.

The examination action is work, examination, or the like to be performed by an examiner. Examples of the examination action include imaging of the ultrasound image, display of the ultrasound image, detection of an abnormality shown in the ultrasound image, display of the abnormality, display and creation of a report, measurement, adjustment of image quality, and the like.

The workflow defines an order in which each examination action is to be performed. For example, it is defined in the workflow that the imaging of the ultrasound image, the detection of the abnormality shown in the ultrasound image, the measurement of the abnormality, and the creation of the report are performed in this order. To describe the imaging of the ultrasound image in detail, the workflow defines a plurality of cross-sections (that is, examination cross-sections), and a plurality of regions (that is, examination regions) imaged by the ultrasonic wave, and an order of imaging each examination cross-section and each examination region.

For example, a typical workflow is determined in advance for each diagnosis region (for example, abdomen, blood vessel, neck, urinary organ, and the like) and for each clinical department (for example, obstetrics and the like). Information indicating a workflow for each diagnosis region or each clinical department is stored in the analysis unit 20, a storage unit of the ultrasonic imaging apparatus, or the like. Further, the information indicating the workflow may be transmitted to the ultrasonic imaging apparatus via a communication path such as a network.

The imaging scene is, for example, an examination action currently performed in the workflow, a scene of the examination currently performed, or a situation of a current examination. Specific examples of the imaging scene include imaging of the examination cross-section and the examination region by the ultrasonic wave, imaging of a site by the ultrasonic wave, and detection of the abnormality shown in the ultrasound image. For example, in a case where a certain examination cross-section is imaged by the ultrasonic wave, imaging the examination cross-section corresponds to an example of the imaging scene. The same applies to the imaging of the site and the detection of the abnormality.

The specifying processing is, for example, identification processing of the ultrasound image, identification processing of the examination cross-section or the examination region, specifying processing of a site imaged in the ultrasound image, detection processing of the abnormality shown in the ultrasound image, or the like.

The analysis unit 20 applies the plurality of types of specifying processing to the ultrasound image to specify the imaging scene. For example, the analysis unit 20 applies processing of identifying the examination cross-section to the ultrasound image, thereby specifying the examination cross-section currently being imaged by the ultrasonic wave, and specifying as the imaging scene that the examination cross-section is imaged. As described above, the order in which the examination actions are to be performed is defined in the workflow. For the imaging of the ultrasound image, an order in which the examination cross-section is imaged is defined. By specifying the examination cross-section currently being imaged by the ultrasonic wave, the examination action (that is, the imaging scene) currently being performed in the workflow is specified.

For example, artificial intelligence (AI) or machine learning is used for the specifying processing. Different artificial intelligence or machine learning may be used for each specifying processing. No limitation limitation is imposed on the type of artificial intelligence or machine learning to be used, and any algorithm or model may be used. For example, a convolutional neural network (CNN), a recurrent neural network (RNN), a generative adversarial network (GAN), a linear model, random forest and decision tree learning, a support vector machine (SVM), an ensemble classifier, or another algorithm is used. In addition, an algorithm that does not require learning such as pattern matching such as template matching, correlation coefficient, or similarity calculation may be used for the specifying processing.

Further, the analysis unit 20 calculates the accuracy of specifying the imaging scene (that is, the degree of certainty of the specified imaging scene). For example, the analysis unit 20 performs the specifying processing using machine learning and calculates the accuracy of specifying using the machine learning. The analysis unit 20 calculates the accuracy for each specifying processing. For example, the analysis unit 20 applies the processing of identifying the examination cross-section to the ultrasound image, thereby identifying the examination cross-section and calculating accuracy of identifying the examination cross-section. In addition, the analysis unit 20 applies processing of identifying a site to the ultrasound image, thereby specifying a site imaged by the ultrasonic wave and calculating accuracy of specifying the site. The same applies to other specific processing.

The analysis unit 20 determines the examination action to be performed next in the workflow on the basis of the result of the specifying processing. For example, the analysis unit 20 determines the examination action to be performed next on the basis of the imaging scene specified for each specifying processing and the accuracy of specifying for each specifying processing.

For example, in the workflow, in a case where the “measurement” is defined as the examination action to be performed next after imaging of a certain examination cross-section, when the imaging of the examination cross-section is specified as the imaging scene, the “measurement” is determined as the examination action to be performed next. That is, since the examination action (that is, the imaging scene) currently being performed is the imaging of the examination cross-section, the “measurement” is determined as the examination action to be performed next.

An execution unit 22 performs the examination action to be performed next as determined by the analysis unit 20 or performs processing related to the examination action to be performed next. The processing related to the examination action is, for example, displaying information for performing the examination action or displaying information prompting the examiner to perform the examination action.

The control unit 24 controls operation of each unit of the ultrasonic imaging apparatus.

The input unit 26 is a device for a user to input to the ultrasonic imaging apparatus conditions, commands, and the like necessary for imaging. For example, the input unit 26 is an operation panel, a switch, a button, a keyboard, a mouse, a trackball, a joystick, or the like.

The ultrasonic imaging apparatus includes the storage unit (not illustrated). The storage unit is a device constituting one or more storage areas for storing data. The storage unit is, for example, a hard disk drive (HDD), a solid state drive (SSD), any of various memories (for example, RAM, DRAM, ROM, or the like), other storage devices (for example, an optical disk or the like), or a combination thereof. For example, the ultrasound image data, information indicating the workflow of the ultrasonic examination, information indicating imaging conditions, and the like are stored in the storage unit.

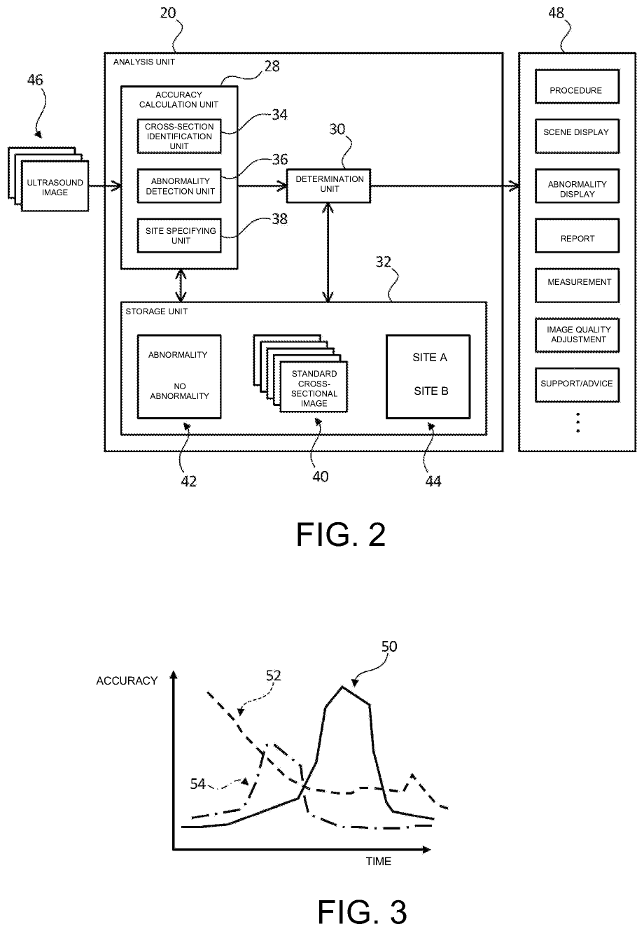

Hereinafter, the analysis unit 20 will be described in detail with reference to FIG. 2. FIG. 2 is a block diagram illustrating the analysis unit 20.

The analysis unit 20 includes an accuracy calculation unit 28, a determination unit 30, and a storage unit 32.

The accuracy calculation unit 28 specifies the imaging scene by applying the plurality of types of specifying processing to the ultrasound image, and calculates the accuracy of specifying the imaging scene for each specifying processing. As described above, artificial intelligence, machine learning, or an accuracy calculation algorithm that does not require learning such as pattern matching, or similarity calculation is used for the specifying processing.

The determination unit 30 determines the examination action to be performed next on the basis of a calculation result (for example, the imaging scene specified for each specifying processing and the accuracy of specifying for each specifying processing) by the accuracy calculation unit 28. The determination unit 30 determines the examination action to be performed next by analyzing the specified imaging scene and the accuracy. For example, the determination unit 30 determines the examination action to be performed next by referring to a database of the workflow or applying pattern matching.

The storage unit 32 is a storage device for storing data used for processing of specifying the imaging scene and data used for determination of the examination action to be performed next. The information indicating the workflow for each diagnosis region or each clinical department may be stored in the storage unit 32.

The accuracy calculation unit 28 includes, for example, a cross-section identification unit 34, an abnormality detection unit 36, and a site specifying unit 38.

The cross-section identification unit 34 applies cross-section identification processing to the ultrasound image to identify the examination cross-section imaged by the ultrasonic wave.

The storage unit 32 stores a plurality of standard cross-sectional images 40 (for example, B-mode images) for identifying the examination cross-section. For example, one or more standard cross-sectional images 40 are prepared in advance for each diagnosis region and stored in the storage unit 32. The standard cross-sectional image 40 is the ultrasound image obtained by imaging a standard examination cross-section with the ultrasonic wave. The standard examination cross-section is, for example, a cross-section to be imaged in the examination, a representative cross-section, or the like. The standard cross-sectional image 40 is an image from which the standard examination cross-section can be identified.

The cross-section identification unit 34 compares an ultrasound image 46 (for example, the B-mode image) generated by transmitting and receiving the ultrasonic wave with the plurality of standard cross-sectional images 40 (that is, a plurality of standard B-mode images) stored in the storage unit 32, to identify the examination cross-section in which the ultrasonic wave is transmitted and received (that is, the examination cross-section in which the ultrasound image 46 is obtained), and calculates the accuracy of identifying the examination cross-section as the accuracy of specifying the imaging scene.

The abnormality detection unit 36 detects the abnormality shown in the ultrasound image by applying abnormality detection processing to the ultrasound image.

The storage unit 32 stores information 42 on the abnormality shown in the ultrasound image. The information 42 on the abnormality is, for example, an image of an abnormal object (for example, a tumor or the like) shown in the ultrasound image, information indicating a place or a position where the abnormality occurs in the diagnosis region, information indicating a shape or a size of the abnormal object, information indicating shading of the abnormal object, and the like.

The abnormality detection unit 36 compares the ultrasound image 46 (for example, the B-mode image) generated by transmitting and receiving the ultrasonic wave with the information 42 on the abnormality, to detect the abnormality from the ultrasound image 46 (for example, identify the tumor or the like) or determine the presence or absence of the abnormality. In addition, the abnormality detection unit 36 calculates accuracy of detecting the abnormality as the accuracy of specifying the imaging scene.

The site specifying unit 38 specifies a site shown in the ultrasound image by applying site specifying processing to the ultrasound image.

The storage unit 32 stores information 44 on the site shown in the ultrasound image. The information 44 on the site is, for example, information indicating a position, a size, a shape, and shading of the site shown in the ultrasound image.

The site specifying unit 38 compares the ultrasound image 46 (for example, the B-mode image) generated by transmitting and receiving the ultrasonic wave with the information 44 on the site, to specify the site shown in the ultrasound image. In addition, the site specifying unit 38 calculates the accuracy of specifying the site as the accuracy of specifying the imaging scene.

FIG. 2 illustrates candidates of the examination action to be performed next as indicated by reference numeral 48. For example, display of a workflow procedure, display of the imaging scene (for example, the display of the ultrasound image), display of the abnormality detected, the display and creation of the report, the measurement of the abnormality, the adjustment of the image quality of the ultrasound image displayed, display of support and advice to the examiner, and the like are illustrated as examples of the examination action to be performed next. For example, the determination unit 30 determines one or more examination actions from among the candidates of the examination action as the examination action to be performed next.

Specific examples of the examination action to be performed next include the following examination actions.
A body mark is displayed or information is associated with the ultrasound image.
A body mark reflecting the presence or absence of abnormality is displayed or information is associated with the ultrasound image.
Annotation reflecting the presence or absence of abnormality is displayed or information is associated with the ultrasound image.
A probe mark is displayed.
A region of interest (ROI) is enlarged and displayed.
An analysis result of deviation from the standard cross-section is displayed.
Measurement is performed when abnormality is detected.
A report is automatically created, or the ultrasound image is inserted into the report.
Measurement selection.

Specifically, implementation of IMT/NT (thickening of back of fetal neck, indication of chromosomal abnormalities)/Simpson method.

Doppler automated measurement (A sample gate is placed near a valve. When the mitral valve or aortic valve is detected, the sample gate is disposed at the detected position.)

Measurement of head-gluteal length, head length, and circumferential length of belly. Measurement of femoral length.
Image adjustment.

Specifically, adjustment of a set of various filter parameters such as band pass filter (BPF), gain curve, gamma curve, transmission focus, and the like.
When a target examination cross-section is imaged, freeze function is executed.
After the freeze function is executed, the examination cross-section with the highest accuracy is selected.
The freeze function is executed when abnormality such as tumor is detected.
When the target examination cross-section is imaged, the ultrasound image of the examination cross-section is stored in the storage unit.
Feedback processing.

Note that at least one of the calculation result by the accuracy calculation unit 28 and a result determined on the basis of the calculation result may be used as feedback to determine the examination action to be performed next. That is, from the calculation result and the result determined on the basis of the calculation result, the calculation result itself may be used, the result determined on the basis of the calculation result may be used, or both the calculation result and the result determined on the basis of the calculation result may be used. For example, information indicating the result (for example, the examination action to be performed next) determined on the basis of the calculation result by the accuracy calculation unit 28 is stored in the storage unit 32 as feedback information. The determination unit 30 may determine the examination action to be performed next with reference to the information indicating the result as well.

In addition, information indicating the examination action which has been actually performed next by the examiner may be stored in the storage unit 32 as the feedback information, and the determination unit 30 may determine the examination action to be performed next with reference to the information as well.

The signal processing unit 14, the image processing unit 16, the analysis unit 20, the execution unit 22, and the control unit 24 can be implemented using, for example, hardware resources such as a processor and an electronic circuit, and a device such as a memory may be used as necessary in the implementation. Further, the signal processing unit 14, the image processing unit 16, the analysis unit 20, the execution unit 22, and the control unit 24 may be implemented by, for example, a computer. That is, all or a part of the signal processing unit 14, the image processing unit 16, the analysis unit 20, the execution unit 22, and the control unit 24 may be implemented by cooperation between hardware resources such as a central processing unit (CPU) and a memory included in the computer, and software (a program) that defines an operation of the CPU and the like. The program is stored in the storage unit of the ultrasonic imaging apparatus or another storage device via a recording medium such as a CD or a DVD or via the communication path such as the network. As another example, the signal processing unit 14, the image processing unit 16, the analysis unit 20, the execution unit 22, and the control unit 24 may be implemented by a digital signal processor (DSP), an application specific integrated circuit (ASIC), a field programmable gate array (FPGA), or the like. Of course, a graphics processing unit (GPU) or the like may be used. The signal processing unit 14, the image processing unit 16, the analysis unit 20, the execution unit 22, and the control unit 24 may be implemented by a single device, or each function of each of the signal processing unit 14, the image processing unit 16, the analysis unit 20, the execution unit 22, and the control unit 24 may be implemented by one or more devices.

Hereinafter, a specific example of processing by the accuracy calculation unit 28 and the determination unit 30 will be described. Here, as an example, a specific example for each diagnosis region will be described.

### Specific Example 1: Abdomen

In screening of an abdomen, particularly a digestive region, a liver, a kidney, a spleen, a pancreas, and the like are imaged. For example, in a protocol of abdominal ultrasonic examination regarding an examination room, a comprehensive medical examination, or the like, 20 or more standard cross-sectional images (corresponding to the examples of the imaging scene) are defined. The number of standard cross-sectional images varies depending on the country or region, and is about 50 in a case where the number is large. The 20 or more standard cross-sectional images are examples of the standard cross-sectional image 40 described above.

For example, the accuracy calculation unit 28 determines which of the 20 or more standard cross-sectional images described above matches the image of the cross-section (that is, the examination cross-section) currently being imaged by the ultrasonic imaging apparatus, and calculates accuracy of the determination of matching (that is, accuracy of identifying the cross-section). For example, the accuracy calculation unit 28 compares the standard cross-sectional image with the image of the currently imaged cross-section for each standard cross-sectional image, to calculate matching degree for each standard cross-sectional image. The matching degree corresponds to an example of the accuracy of identifying the cross-section.

The determination unit 30 determines the examination action to be performed next on the basis of a result of identification by the accuracy calculation unit 28. For example, the determination unit 30 determines execution of an abdominal routine examination in the examination room, or determines execution of a part or all of report display in an abdominal examination in a comprehensive medical examination or the like.

For example, in a case where the matching degree between the image of the currently imaged cross-section and the standard cross-sectional image of the spleen is 80% or more, the determination unit 30 determines the examination action to be performed next on the basis of a workflow for examining the spleen. For example, the determination unit determines attachment and arrangement of the ultrasound image on the report as the examination action to be performed next. The execution unit 22 performs the examination action determined by the determination unit 30. In this example, the execution unit 22 displays the report on the display unit 18, and displays the image of the currently imaged cross-section at a place where a spleen image is placed in the report. Thus, it is possible to save time and effort for the examiner to manually select the spleen image from a stored image group. As a result, the examination time is reduced and the workflow is improved. In addition, an alert indicating that the cross-section to be imaged has not been imaged may be output.

The accuracy calculation unit 28 may calculate the matching degree with the standard cross-sectional image and the accuracy of detecting the abnormal object. That is, the accuracy calculation unit 28 calculates the matching degree between the image of the currently imaged cross-section and the standard cross-sectional image. Further, the accuracy calculation unit 28 detects the abnormal object from the image of the currently imaged cross-section, and calculates the accuracy of detecting the abnormal object. In this case, the determination unit 30 determines the examination action to be performed next on the basis of the matching degree with the standard cross-sectional image and the accuracy of detecting the abnormal object.

For example, degree of deviation from an image of the liver of a healthy person without cancer is used. For example, in a case where the matching degree between the image of the currently imaged cross-section and the standard cross-sectional image with the liver is 80% or more, the abnormal object is detected from the image of the currently imaged cross-section, and the accuracy of detecting the abnormal object is 80% or more, the determination unit 30 determines an examination mode of the tumor as the examination action to be performed next on the basis of a predetermined determination criterion. In this case, the execution unit 22 automatically activates the examination mode of the tumor without receiving an activation instruction from the examiner. The execution unit 22 may cause the display unit 18 to display information (for example, information indicating an alert) prompting the examiner to determine whether to activate the examination mode.

### Specific Example 2: Blood Vessel

In a blood vessel examination, the workflow can be improved, for example, regarding determination of the presence or absence of plaque in a carotid artery.

As with the abdomen, there are about 5 to 10 (greater than or equal to 20 in some cases) standard cross-sectional images of the carotid artery. For the carotid artery as well, accuracy of the matching degree is calculated for each standard cross-sectional image. Based on the calculation result, the attachment of the image on the report or another examination action is determined as the examination action to be performed next. Then, the determined examination action is performed. Thus, manual operation of the examiner is omitted, and the workflow can be improved.

Further, the accuracy calculation unit 28 may calculate smoothness of a blood vessel wall on the basis of a change in brightness of the blood vessel shown in the image of the currently imaged cross-section, a magnitude of the brightness, or the like. Further, the accuracy calculation unit 28 may calculate a probability that the plaque (an example of the abnormal object) is present on the blood vessel wall by calculating the degree of deviation from a normal blood vessel without the plaque.

For example, in a case where accuracy that a vascular bifurcation is shown in the image of the currently imaged cross-section is 80% or more, and accuracy that the abnormal object such as the plaque is present on the blood vessel wall is 80% or more, the determination unit 30 determines execution of measurement mode of IMT (blood vessel wall thickness) as the examination action to be performed next on the basis of a predetermined determination criterion. In this case, the execution unit 22 automatically activates the measurement mode of IMT or causes the display unit 18 to display information (for example, information indicating an alert) prompting the examiner to determine whether to activate the measurement mode. The execution unit 22 may cause the display unit 18 to display information prompting the examiner to newly add a plaque protocol to the workflow under examination. Thus, more accurate examination can be performed.

In addition, information indicating a measurement result of IMT may be input to the analysis unit 20 and stored in the storage unit 32. For example, in a case where a thickness of the blood vessel obtained by IMT measurement is greater than a predetermined thickness, the execution unit 22 may propose to the examiner a predetermined recommended finding such as “there is a possibility of plaque.” The execution unit 22 may insert the recommended finding into the report, or may cause the display unit 18 to display information (for example, information indicating an alert) prompting the examiner to insert the recommended finding into the report. Since the examiner does not need to manually perform these operations, it is possible to automate the operations and reduce operation time, thereby improving the workflow.

### Specific Example 3: Circulatory Organ

In a cardiovascular field, there are many special measurements in routine examinations such as ejection fraction (EF) measurement and speckle tracking of a heart wall. Therefore, the present embodiment is effective for determining the examination action to be performed next.

In the examination of the heart, examination items for each standard cross-section are defined. For example, the accuracy calculation unit 28 calculates, as the accuracy, the matching degree between the image of the cross-section being imaged and the standard cross-sectional image such as an apical 4 chamber view (A4C). On the basis of the calculation result, the determination unit 30 determines an examination action for measuring volumes of the right ventricle, left ventricle, right atrium, and left atrium or an examination action for measuring a vascular system or a valve as an examination action to be performed next.

Further, the accuracy calculation unit 28 may calculate the matching degree between the image of the currently imaged cross-section and the standard cross-sectional image, and accuracy as to whether the cross-section suitable for each measurement is visualized. For example, in the case of measuring the left ventricle, if dropout of echo is large on a left ventricular wall surface, the left ventricle is overestimated. In this case, the accuracy calculation unit 28 may calculate accuracy of visualizing the left ventricular wall surface, or the like, as accuracy of the imaging scene. On the basis of the matching degree and the accuracy of visualization, the determination unit 30 determines, as the examination action to be performed next, an examination action of automatically measuring a site other than a left ventricular volume and an examination action of notifying the examiner of a message such as “there is a possibility that a left ventricular ejection amount cannot be accurately measured” for the left ventricular ejection amount. This makes it possible to perform a more accurate and more rapid routine examination in the cardiovascular field.

### Specific Example 4: Obstetrics

In an obstetric examination, similarly to the examination of the circulatory organ, there are many examination items, and thus the present embodiment is effective for determining the examination action to be performed next.

As a specific example, an examination of a fetus will be described as an example. The accuracy calculation unit 28 calculates the matching degree between the image of the currently imaged cross-section by transmitting and receiving the ultrasonic wave to and from the fetus and the standard cross-sectional image of fetal ultrasonic examination. The determination unit 30 determines, as the examination action to be performed next, a measurement mode for measuring a crown-rump length (CRL), a biparietal diameter (BPD), a femur length (FL), and the like, on the basis of the matching degree. The execution unit 22 performs the determined measurement mode.

The accuracy calculation unit 28 may calculate an abnormal site, features peculiar to a congenital disease, or the like as the degree of deviation from normal from the image of the currently imaged cross-section. On the basis of the matching degree with the standard cross-sectional image and the features, the determination unit 30 determines, as the examination action to be performed next, an examination action including a branch in a case where there is the abnormality and in a case where there is no abnormality.

As described above, even in obstetrics, a more accurate and more rapid fetal screening examination can be performed.

### Specific Example 5: Mammary Gland

In the examination of mammary gland, the tumor such as breast cancer is mainly determined. Whether the cross-section suitable for the determination is imaged is determined by the accuracy calculation unit 28. On the basis of the determination result, the determination unit 30 determines the examination action for determining the presence or absence of the breast cancer as the examination action to be performed next.

For example, the accuracy calculation unit 28 determines whether the image quality of the image of the currently imaged cross-section is sufficient to determine the presence or absence of the breast cancer, and calculates accuracy of the image quality. The accuracy calculation unit 28 calculates the accuracy of the image quality on the basis of various parameters such as an area of a breast region shown in the image of the currently imaged cross-section, contrast of the image, and an invalid area. For example, in a case where the accuracy is 80% or more, the determination unit 30 determines the examination action for determining the presence or absence of the breast cancer as the examination action to be performed next.

The accuracy calculation unit 28 may calculate, as the accuracy of detecting the abnormal site, a probability that the abnormal site different from a normal site is shown in the image of the currently imaged cross-section.

The determination unit 30 receives a result calculated by the accuracy calculation unit 28 as described above, and determines an automatic measurement application for breast cancer detection as the examination action to be performed next on the basis of the predetermined determination criterion. In this case, the execution unit 22 automatically starts the automatic measurement application.

As another example, the determination unit 30 may determine processing of displaying a message (for example, an alert) such as “there is a possibility of the abnormal site” as the examination action to be performed next. In this case, the execution unit 22 causes the display unit 18 to display the message.

As described above, in mammary gland examination, a more accurate and more rapid breast cancer examination can be performed. In addition, a burden imposed on the examiner such as the technician can be reduced.

(Display Example of Identification Result of Examination Cross-Section)

Hereinafter, a display example of a result of identification by the cross-section identification unit 34 will be described with reference to FIGS. 3 to 10.

FIG. 3 is a graph illustrating the accuracy of identifying the examination cross-section. The horizontal axis indicates time, and the vertical axis indicates accuracy of identification.

Graphs 50, 52, and 54 illustrate temporal changes in accuracy. The graph 50 illustrates the temporal change in accuracy that the currently imaged cross-section is an examination cross-section A. The graph 52 illustrates the temporal change in accuracy that the currently imaged cross-section is an examination cross-section B. The graph 54 illustrates the temporal change in accuracy that the currently imaged cross-section is an examination cross-section C.

When the examiner changes a position and direction of the ultrasonic probe 10, the cross-section imaged by the ultrasonic wave is changed. Thus, for example, as illustrated in FIG. 3, the accuracy of each examination cross-section changes over time.

The image processing unit 16 may display each graph illustrated in FIG. 3 on the display unit 18. Thus, the examiner can recognize which cross-section is currently imaged, and can check the accuracy.

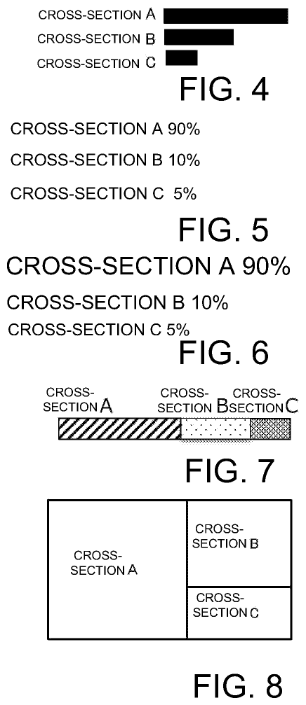

As illustrated in FIG. 4, the accuracy of each examination cross-section may be represented by a bar. A length of the bar corresponds to the accuracy, and the longer the bar, the higher the accuracy.

As illustrated in FIGS. 5 and 6, the accuracy of each examination cross-section may be displayed as a numerical value. In an example illustrated in FIG. 5, sizes of character strings indicating accuracies of examination cross-sections are the same. In an example illustrated in FIG. 6, the size of the character string reflects the degree of accuracy. The larger the character string, the higher the accuracy.

As illustrated in FIG. 7, the accuracy of each examination cross-section may be represented by the bar. In an example illustrated in FIG. 7, bars corresponding to the accuracies of the examination cross-sections are displayed in series.

As illustrated in FIG. 8, the accuracy of each examination cross-section may be represented by a two-dimensional figure. A size (that is, an area) of the figure corresponds to the accuracy, and the larger the area, the higher the accuracy.

The above-described bar, character string, figure, or the like is displayed on the display unit 18. For example, each figure illustrated in FIG. 8 is displayed on the display unit 18. When the examiner selects a certain figure (for example, the figure of the examination cross-section A), information indicating the examination cross-section A (for example, information indicating the site) may be linked to the image of the currently imaged cross-section.

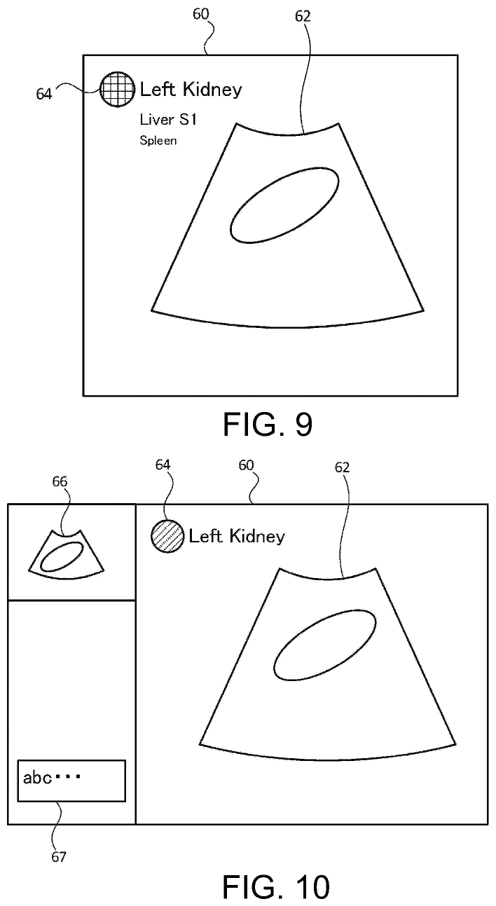

The image processing unit 16 may cause the display unit 18 to display the image of the currently imaged cross-section and the information indicating the accuracy of the examination cross-section. FIG. 9 illustrates a display example thereof. An ultrasound image 62 such as the B-mode image is displayed on a screen 60 of the display unit 18. In addition, the character string indicating the identified examination cross-section and an image 64 indicating the accuracy of the identification are displayed on the screen 60. Here, as an example, an examination cross-section “Left Kidney” is identified, and its accuracy is represented by the image 64. For example, the accuracy is represented by the color, size, and shape of the image 64. For example, green image 64 represents high accuracy, yellow image 64 represents medium accuracy, and red image 64 represents low accuracy. Of course, the accuracy may be represented by a numerical value. In addition, information indicating a candidate (for example, “Liver”, “Spleen”, and the like) of the examination cross-section other than the examination cross-section “Left Kidney” may be displayed on the screen 60.

By displaying the ultrasound image 62 and the image 64 indicating the accuracy, the examiner can determine how much the image of the currently imaged cross-section matches the standard cross-sectional image. Further, these pieces of information are useful for education and training of the examiner. Further, the examiner can check whether the abnormal object is shown in the ultrasound image 62 by checking a difference between the standard cross-sectional image and the ultrasound image 62. Furthermore, displaying these pieces of information can also be a reminder to the examiner.

As illustrated in FIG. 10, a standard cross-sectional image 66 may be displayed on the screen 60 together with the ultrasound image 62 of the currently imaged cross-section. In this way, the examiner can perform the examination while comparing the standard cross-sectional image 66 and the ultrasound image 62. In addition, an alert or a comment 67 prompting the examiner to make a next determination, supporting insertion, or suggesting a possibility that the cross-section to be imaged is not imaged may be displayed together on the screen 60.

(Display of Body Mark)

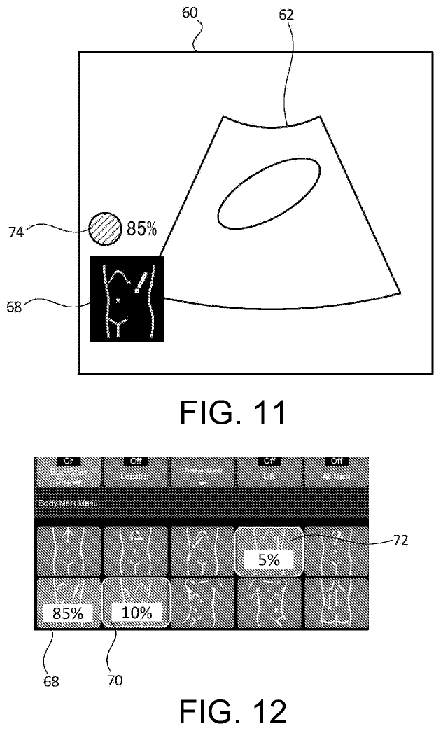

Hereinafter, processing of setting the body mark in the ultrasound image will be described with reference to FIGS. 11 and 12. FIG. 11 is a diagram illustrating a display example of the ultrasound image. FIG. 12 is a diagram illustrating the body mark.

Here, as an example, the examination action to be performed next is to set the body mark in the ultrasound image.

The accuracy calculation unit 28 calculates the matching degree between the image of the currently imaged cross-section and each standard cross-sectional image as the accuracy of the imaging scene.

The determination unit 30 specifies the body mark associated with the standard cross-section having a calculated matching degree greater than or equal to a threshold. For example, the standard cross-section and the body mark representing imaging of the standard cross-section are associated in advance for each standard cross-section, and information indicating the association (for example, an association table or the like) is stored in the storage unit 32. The determination unit 30 specifies the body mark associated with the standard cross-section having a matching degree greater than or equal to the threshold in the association table, and determines the setting of the body mark as the examination action to be performed next.

For example, the execution unit 22 causes the display unit 18 to display the body mark determined by the determination unit 30 together with the image of the currently imaged cross-section.

FIG. 11 illustrates a display example of the body mark. The ultrasound image 62, a body mark 68, and an image 74 are displayed on the screen 60. The ultrasound image 62 is the image of the currently imaged cross-section. The body mark 68 is the body mark associated with the standard cross-section having a matching degree greater than or equal to the threshold. The image 74 is an image indicating accuracy of the body mark 68. For example, the image 74 is displayed in color corresponding to the accuracy. Here, as an example, the accuracy is 85%. That is, the body mark 68 with 85% accuracy is displayed.

FIG. 12 illustrates another display example of the body mark. For example, the execution unit 22 causes the display unit 18 to display a plurality of candidates for the body mark. In addition, the execution unit 22 causes the display unit 18 to display the information indicating the accuracy for each body mark. The accuracy here is a likelihood that the body mark is suitable for a body mark corresponding to the currently imaged cross-section; in other words, corresponds to the matching degree between the currently imaged cross-section and the standard cross-section.

For example, the accuracy of the body mark 68 is 85%, the accuracy of a body mark 70 is 10%, and the accuracy of a body mark 72 is 5%.

When the examiner selects a target body mark from a list of candidates illustrated in FIG. 12, the image processing unit 16 causes the display unit 18 to display the selected body mark together with the ultrasound image 62. For example, since the accuracy is displayed, the examiner can select the body mark with reference to the displayed accuracy.

In addition, a list of names of candidate cross-sections may be displayed, or a list of probe marks may be displayed. At least one of the body mark, the probe mark, and the name of the cross-section may be displayed.

(Selection of Optimal Image)

When an optimum image is captured in relation to the standard cross-sectional image, the optimum image may be selected and displayed. The optimum image may be, for example, an ultrasound image having a matching degree with the standard cross-sectional image greater than or equal to the threshold among a plurality of currently imaged ultrasound images, or an ultrasound image having the highest matching degree. For example, when the freeze function is executed, the optimum image is displayed.

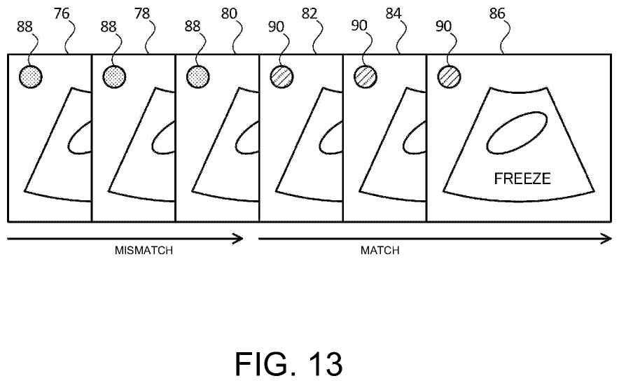

Hereinafter, selection of the optimum image will be described with reference to FIG. 13. FIG. 13 illustrates ultrasound images 76 to 86. The ultrasound images 76 to 86 are respectively the B-mode images of the currently imaged cross-section. The ultrasound images are imaged in the order of the ultrasound images 76 to 86.

The accuracy calculation unit 28 calculates the matching degree between the image of the currently imaged cross-section and the standard cross-sectional image. The standard cross-sectional image is determined on the basis of the diagnosis region or the clinical department. For example, in a case where the standard cross-sectional image of the spleen is designated, the accuracy calculation unit 28 calculates the matching degree between the image of the currently imaged cross-section and the standard cross-sectional image of the spleen.

Each ultrasound image of the ultrasound images 76 to 86 is sequentially imaged, and the accuracy calculation unit 28 sequentially calculates the matching degree between the captured ultrasound image and the standard cross-sectional image of the spleen.

In an example illustrated in FIG. 13, the ultrasound images 76 to 80 do not match the standard cross-sectional image. For example, the matching degree between the ultrasound image 76 and the standard cross-sectional image of the spleen is less than the threshold. The same applies to the ultrasound images 78 and 80. An image 88 is illustrated superimposed on each of the ultrasound images 76 to 80. The image 88 is an icon, a mark, or the like indicating that the matching degree is low.

On the other hand, the ultrasound images 82 to 86 match the standard cross-sectional image. For example, the matching degree between the ultrasound image 82 and the standard cross-sectional image of the spleen is greater than or equal to the threshold. The same applies to the ultrasound images 84 and 86. An image 90 is illustrated superimposed on each of the ultrasound images 82 to 86. The image 90 is an icon, a mark, or the like indicating that the matching degree is high.

For example, in a case where a plurality of ultrasound images having a matching degree greater than or equal to the threshold are consecutively imaged, and the number of consecutive images is greater than or equal to a number threshold, the determination unit determines the freeze function as the examination action to be performed next. The execution unit 22 automatically executes the freeze function.

For example, at the time when the ultrasound image 86 is captured, the execution unit 22 executes the freeze function in a case where the number of consecutive images is greater than or equal to the number threshold. Thus, the ultrasound image 86 of the currently imaged cross-section is displayed in a stationary state on the display unit 18. The examiner can observe the ultrasound image 86 that matches the standard cross-sectional image in the stationary state.

(Search of Ultrasound Image)

The accuracy calculation unit 28 may search for the optimum image from the plurality of ultrasound images that have already been imaged and stored in the storage unit. For example, the execution unit 22 displays the searched ultrasound image on the display unit 18.

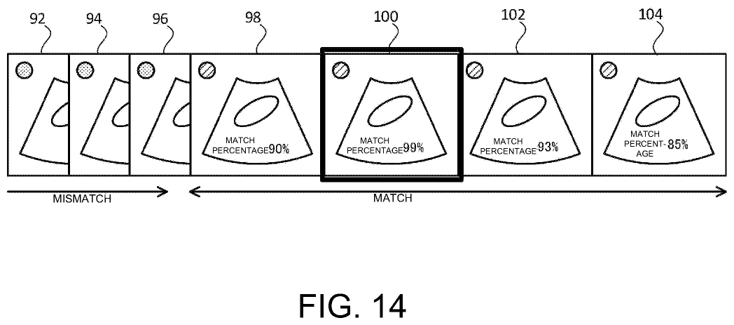

The search of the ultrasound image will be described with reference to FIG. 14. FIG. 14 illustrates ultrasound images 92 to 104. The ultrasound images 92 to 104 are the B-mode images that have already been imaged and stored in the storage unit. For example, when each of the ultrasound images 92 to 104 is captured, the freeze function is executed, whereby the ultrasound images 92 to 104 are stored in the storage unit.

The accuracy calculation unit 28 calculates the matching degree between each of the ultrasound images 92 to 104 and the standard cross-sectional image. As described above, the standard cross-sectional image is determined on the basis of the diagnosis region or the clinical department.

In an example illustrated in FIG. 14, the ultrasound images 92 to 96 do not match the standard cross-sectional image. For example, the matching degree of each of the ultrasound images 92 to 96 is less than the threshold.

On the other hand, the ultrasound images 98 to 104 match the standard cross-sectional image. For example, the matching degree of each of the ultrasound images 98 to 104 is greater than or equal to the threshold. Specifically, the matching degree of the ultrasound image 98 is 90%, the matching degree of the ultrasound image 100 is 99%, the matching degree of the ultrasound image 102 is 93%, and the matching degree of the ultrasound image 104 is 85%.

In the example illustrated in FIG. 14, since the matching degree of the ultrasound image 100 is the highest, the accuracy calculation unit 28 selects the ultrasound image 100. The execution unit 22 displays the selected ultrasound image 100 on the display unit 18. Of course, the execution unit 22 may cause the display unit 18 to display all or some of the ultrasound images having a matching degree greater than or equal to the threshold.

(Automatic Image Quality Adjustment)

The image quality of the ultrasound image may be automatically adjusted to match the features shown in the ultrasound image. For example, the determination unit 30 determines a set value (for example, a parameter) for image quality adjustment in real time for the image of the currently imaged cross-section. As another example, the determination unit 30 determines the set value for the image quality adjustment for an image captured when the freeze function is executed. As still another example, the determination unit 30 may determine the set value for the image quality adjustment according to an instruction of the examiner. For example, when the examiner determines an image to be stored and presses a button for instructing the image quality adjustment, the determination unit 30 determines the set value for the image quality adjustment.

Automatic image quality adjustment will be described by taking an abdomen region as an example. The set value for the image quality adjustment is determined in advance. The accuracy calculation unit 28 determines whether a target site (for example, an organ to be imaged or the like) is shown in a captured B-mode image. When the target site can be recognized from the B-mode image, the set value for the image quality adjustment is maintained.

In a case where the target site is not shown in the B-mode image or in a case where the organ to be imaged is present in a deep portion, the determination unit 30 selects a set value for the deep portion. The execution unit 22 changes the set value for the image quality adjustment to the set value for the deep portion. Thus, the image quality is adjusted according to the set value for the deep portion even if the examiner does not manually select the set value for the deep portion as the set value for the image quality adjustment.

The image quality adjustment may be automatically performed also in the color Doppler method. For example, in a case where an arterial system of the abdomen is imaged, the determination unit 30 selects a predetermined standard set value. The execution unit 22 maintains the set value for the image quality adjustment at the standard set value.

In a case where a renal blood flow is imaged, the determination unit 30 selects a set value for the kidney. The execution unit 22 changes the set value for the image quality adjustment to the set value for the kidney. Thus, the image quality is adjusted according to the set value for the kidney even if the examiner does not manually select the set value for the kidney as the set value for the image quality adjustment.

(Setting of Measurement Region)

In a case where the measurement is performed on the ultrasound image, the measurement region may be automatically set to match the features shown in the ultrasound image.

For example, the determination unit 30 determines a position and size of a region of interest (ROI) for designating a region to be measured on the basis of the calculation result by the accuracy calculation unit 28.

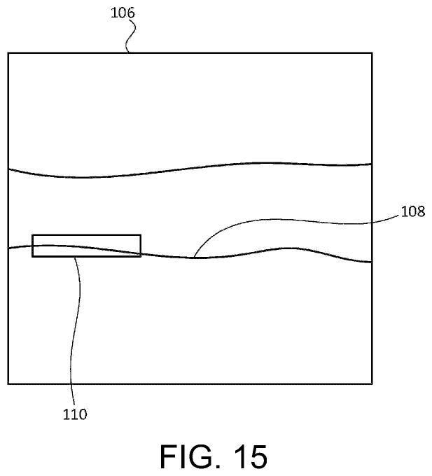

Automatic setting of the ROI will be described by taking IMT measurement of the carotid artery as an example. FIG. 15 illustrates an ultrasound image 106. A carotid artery 108 is displayed on the ultrasound image 106. In addition, an ROI 110 is displayed. The ROI 110 is a figure for designating a region to be subjected to the IMT measurement.

In the IMT measurement of the carotid artery, a position about 1 cm away from a bifurcation of the carotid artery is measured. The accuracy calculation unit 28 specifies that the ultrasound image 106 is an image for IMT measurement, specifies a position of the blood vessel (for example, the carotid artery 108) shown in the ultrasound image 106, and calculates accuracies of specifying them. The determination unit 30 determines the position and size of the ROI 110 on the basis of the calculation result by the accuracy calculation unit 28. The execution unit 22 displays the ROI 110 having the determined size at a position determined by the determination unit 30.

Conventionally, an ROI having a predetermined size is displayed at a predetermined position (for example, in the center of the ultrasound image). Therefore, the examiner needs to move the ROI to the region to be subjected to the IMT measurement and change the size of the ROI. According to the present embodiment, the ROI 110 having a size matching the size of the region is automatically set in the region to be subjected to the IMT measurement. Therefore, it is possible to save time and effort for the examiner to manually set the ROI.

(Attachment of Ultrasound Image on Report)

The determination unit 30 may determine attachment of the ultrasound image on the report as the examination action to be performed next. For example, the determination unit determines, as the examination action to be performed next, processing of attaching to the report the ultrasound image having a matching degree with the standard cross-sectional image greater than or equal to the threshold. The execution unit 22 automatically attaches to the report the ultrasound image having a matching degree with the standard cross-sectional image greater than or equal to the threshold.

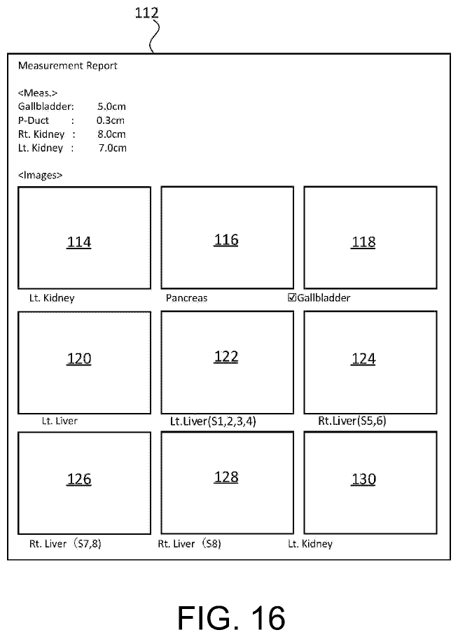

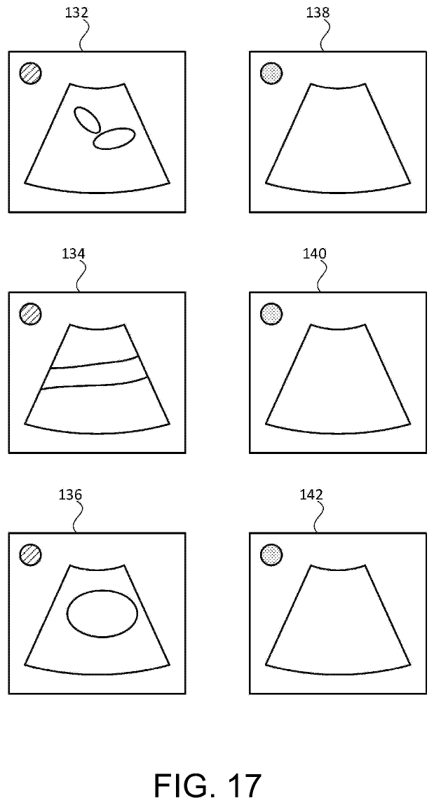

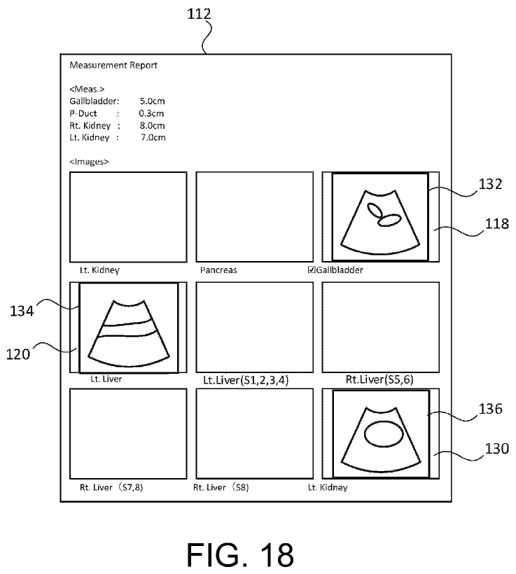

Hereinafter, the processing of attaching the ultrasound image to the report will be described with reference to FIGS. 16 to 18. FIG. 16 illustrates a report 112 to which no ultrasound image is attached. FIG. 17 illustrates ultrasound images 132 to 142. FIG. 18 illustrates the report 112 to which the ultrasound image is attached.

The report 112 is an electronic report, an electronic medical record, or the like. As illustrated in FIG. 16, regions 114 to 130 to which the ultrasound image is attached are determined in the report 112. For example, the ultrasound image to be attached is determined for each region. Specifically, the site, the organ, or the like is associated with each region. For example, the liver is associated with the region 120. That is, the region 120 is a region to which the ultrasound image representing the liver is attached.

Each of the ultrasound images 132, 134, and 136 illustrated in FIG. 17 is an image having a matching degree with the standard cross-sectional image greater than or equal to the threshold. Each of the ultrasound images 138, 140, and 142 is an image having a matching degree with the standard cross-sectional image less than the threshold.

The ultrasound image 132 is an image having a matching degree with a standard cross-sectional image of a gallbladder, that is greater than or equal to the threshold. Therefore, the ultrasound image 132 is an image to be attached to a region of the gallbladder in the report. The ultrasound image 134 is an image having a matching degree with a standard cross-sectional image of the liver, that is greater than or equal to the threshold. Therefore, the ultrasound image 134 is an image to be attached to a region of the liver in the report. The ultrasound image 136 is an image having a matching degree with a standard cross-sectional image of the kidney, that is greater than or equal to the threshold. Therefore, the ultrasound image 136 is an image to be attached to a region of the kidney in the report. These matching degrees are calculated by the accuracy calculation unit 28. The region to which each of the ultrasound images 132, 134, and 136 is attached is specified by the determination unit 30.

FIG. 18 illustrates the report 112 to which the ultrasound images 132, 134, and 136 are attached. The ultrasound image 132 is attached to a region 118 of the gallbladder. The ultrasound image 134 is attached to a region 120 of the liver. The ultrasound image 136 is attached to a region 130 of the kidney. These attachments are performed by the execution unit 22.

For example, the execution unit 22 displays the report 112 on the display unit 18. Further, the execution unit 22 displays the ultrasound image 132 in the region 118, displays the ultrasound image 134 in the region 120, and displays the ultrasound image 136 in the region 130. Furthermore, the execution unit 22 may associate the ultrasound image 132 with the region 118, associate the ultrasound image 134 with the region 120, and associate the ultrasound image 136 with the region 130, to store the report 112 and the ultrasound images 132, 134, and 136 in the storage unit.

Since the ultrasound image is automatically attached to the report as described above, it is possible to save time and effort for the examiner to select and attach the ultrasound images to the report.

Even when an ultrasound image is automatically attached to the report, the examiner can change the attached ultrasound image to another ultrasound image or delete the attached ultrasound image. In addition, the examiner can select the ultrasound image to be attached to the report for a site or organ having a matching degree of the ultrasound image less than the threshold.

When a finding is described in the report 112, the finding is displayed together with the ultrasound image. The execution unit 22 may select a candidate of the finding related to the site or organ shown in the ultrasound image attached to the report 112 from a preset list of findings and display the selected candidate of the finding on the display unit 18. The examiner can select the finding from the candidates.

The execution unit 22 may cause the display unit 18 to display each of a plurality of captured ultrasound images as a thumbnail image. When the examiner selects the thumbnail image to be attached to the report from among the plurality of thumbnail images, the ultrasound image corresponding to the selected thumbnail image is attached to the report. In a case where the ultrasound image having a matching degree with the standard cross-sectional image that is greater than or equal to the threshold is automatically attached to the report, information indicating that the thumbnail image has been selected may be associated with the thumbnail image of the ultrasound image attached to the report. For example, an image or a character string indicating that the thumbnail image has been selected is displayed superimposed on the thumbnail image.

1. An ultrasonic imaging apparatus comprising:
an accuracy calculation unit configured to receive an ultrasound image generated by transmitting and receiving an ultrasonic wave, specify an imaging scene by a plurality of types of specifying processing for specifying the imaging scene of the ultrasound image, and calculate accuracy of specifying the imaging scene for each specifying processing; and
a determination unit configured to determine an examination action to be performed next on the basis of a result of calculation by the accuracy calculation unit.

2. The ultrasonic imaging apparatus according to claim 1, wherein
the plurality of types of specifying processing includes identification processing of a cross-section in which the ultrasonic wave is transmitted and received, and
the accuracy calculation unit compares an image of a predetermined standard cross-section with an image of the cross-section in which the ultrasonic wave is transmitted and received to identify the cross-section in which the ultrasonic wave is transmitted and received, and calculates accuracy of identifying the cross-section as the accuracy of specifying the imaging scene.

3. The ultrasonic imaging apparatus according to claim 2, wherein
the plurality of types of specifying processing further include processing of detecting an abnormality shown in the ultrasound image, and
the accuracy calculation unit further detects the abnormality from the ultrasound image and calculates accuracy of detecting the abnormality as the accuracy of specifying the imaging scene.

4. The ultrasonic imaging apparatus according to claim 3, wherein
the plurality of types of specifying processing further include processing of specifying a site shown in the ultrasound image, and
the accuracy calculation unit further specifies the site shown in the ultrasound image and calculates accuracy of specifying the site as the accuracy of specifying the imaging scene.

5. The ultrasonic imaging apparatus according to claim 4, wherein
at least one of the result of the calculation by the accuracy calculation unit and a result determined on the basis of the result of the calculation is used to determine the examination action to be performed next.

6. The ultrasonic imaging apparatus according to claim 1, wherein
the determination unit determines setting of a body mark to be set in the ultrasound image as the examination action to be performed next.

7. The ultrasonic imaging apparatus according to claim 1, wherein
the determination unit determines attachment of the ultrasound image on a report as the examination action to be performed next.

8. A computer-readable recording medium recording a program for causing a computer to function as:
an accuracy calculation unit configured to receive an ultrasound image generated by transmitting and receiving an ultrasonic wave, to specify an imaging scene by a plurality of types of specifying processing for specifying the imaging scene of the ultrasound image, and to calculate accuracy of specifying the imaging scene for each specifying processing; and
a determination unit configured to determine an examination action to be performed next on the basis of a result of calculation by the accuracy calculation unit.
